(19) United States
(12) Patent Application Publication
(10) Pub. No.: US 2019/0054497 A1
(43) Pub. Date: Feb. 21, 2019

(54) ROTATIONAL NEEDLE

(71) Applicant: Darren Brooks, Chilliwack (CA)

(72) Inventor: Darren Brooks, Chilliwack (CA)

(21) Appl. No.: 15/678,947

(22) Filed: Aug. 16, 2017

(51) Int. Cl. B05C 17/005

(52) U.S. Cl. CPC B05C 17/00593; A61M 2005/3289

(57) ABSTRACT

An apparatus for rotating a needle comprising an inner sleeve securable to a syringe barrel, the inner sleeve having an inner sleeve passage extending therethrough, an outer sleeve rotatably supported by the inner sleeve, the outer sleeve having an outer sleeve passage extending therethrough in fluidic communication with the inner sleeve passage, the outer sleeve having a needle receptacle adapted to receive and operably couple a needle thereon, and a rotational energy-storing device, operably connected to the inner and outer sleeve so as to provide a rotational torque therebetween. A method for rotating a needle comprising rotating the outer sleeve relative to the inner sleeve so as to store rotational energy within the rotational energy-storing device extending between the inner and outer sleeves and releasing the outer sleeve such that the needle rotates relative to the syringe barrel.

### BACKGROUND OF THE INVENTION

### 1. Field of Invention

The present invention relates generally to dental anesthetic needles, and specifically to a rotating dental needle device.

### 2. Description of Related Art

In the dental industry, dental anesthetic is administered to the patient via a dental needle. Disposable needles have greatly increased the consistency of needle sharpness and anesthetic delivery, yet some patients still experience significant pain during injection. The static needle penetrates tissue with blunt force, causing penetration trauma.

In prior art, as illustrated in FIG. 1, a dental syringe 10 includes a needle adapter 12 adapted to engage upon the distal end of a syringe barrel 14 which includes internal threading 16, as is commonly known. A needle (not shown) is adapted to engage upon the needle adapter 12 at external threading 18, with a portion of the needle passing therethrough to an anesthetic cartridge (not shown) contained within the syringe barrel 14, as is commonly known. The external threading 18 is standard in the industry to allow the use of a wide variety of needles. Injection proceeds as is commonly known, with the dentist controlling the location and direction of the needle, as well as the flow of the anesthetic through the needle into the patient. The static needle is manually pushed through the patient's tissues, as is commonly known in the art.

### SUMMARY OF THE INVENTION

According to a first embodiment of the present invention there is disclosed an apparatus for rotating a needle comprising an inner sleeve securable to a syringe barrel, the inner sleeve having an inner sleeve passage extending therethrough, an outer sleeve rotatably supported by the inner sleeve, the outer sleeve having an outer sleeve passage extending therethrough in fluidic communication with the inner sleeve passage, the outer sleeve having a needle receptacle adapted to receive and operably couple a needle thereon, and a rotational energy-storing device, operably connected to the inner and outer sleeve so as to provide a rotational torque therebetween.

The apparatus may further comprise at least one bearing rotatably supporting the outer sleeve on the inner sleeve. The at least one bearing may be selected from a group consisting of roller bearings and bushings.

The inner sleeve may include a stationary nipple extending therefrom wherein the outer sleeve may include a cavity extending thereinto wherein the stationary nipple may be located within the cavity. The at least one bearing may be located around the stationary nipple within the cavity.

The inner sleeve passage and the outer sleeve passage may extend along a central axis through the inner and outer sleeves. The needle receptacle may include a nipple portion at a distal end of the outer sleeve. The nipple portion may include external threading thereon, adapted to receive the needle thereon. The outer sleeve passage of the outer sleeve and the inner sleeve passage of the inner sleeve may be adapted to receive the needle therethrough.

The inner sleeve may include a mounting portion for securing the inner sleeve to the syringe barrel. The mounting portion may include external threading thereon, adapted to be received within the syringe barrel.

The rotational energy storing device may comprise a spiral torsion spring. The spiral torsion spring may include first and second ends. The first end of the spiral torsion spring may be secured to an outer surface of the inner sleeve and the second end of the spiral torsion spring may be secured to an inner surface of the outer sleeve.

The apparatus may further comprise a gear assembly connected to the spiral torsion spring adapted to rotate the outer sleeve relative to the inner sleeve at a predefined rate.

According to a further embodiment of the present invention there is disclosed a method for rotating a needle comprising securing an inner sleeve to a syringe barrel, rotatably supporting an outer sleeve with the inner sleeve and securing a needle to the outer sleeve so as to place a passage through the needle in fluidic communication with a capsule within the syringe barrel. The method further comprises rotating the outer sleeve relative to the inner sleeve so as to store rotational energy within a rotational energy-storing device extending between the inner and outer sleeves and releasing the outer sleeve such that the needle rotates relative to the syringe barrel.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention wherein similar characters of reference denote corresponding parts in each view,

FIG. 1 is a cross sectional view of a prior art syringe.

FIG. 2 is a perspective view of a syringe having a rotational needle device installed thereon according to a first embodiment of the present invention.

FIG. 3 is a cross-sectional view of the rotational needle device of FIG. 2 taken along the line 3-3.

FIG. 4 is a exploded cross-sectional view of the rotational needle device of FIG. 2.

### DETAILED DESCRIPTION

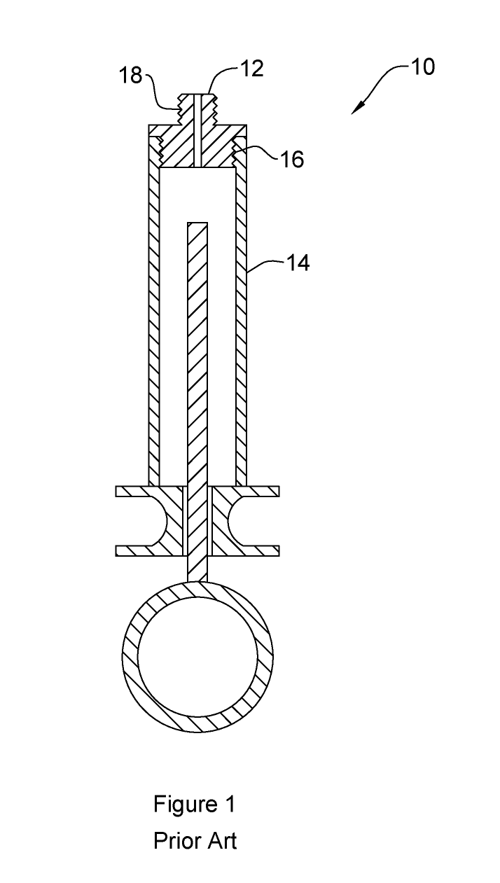

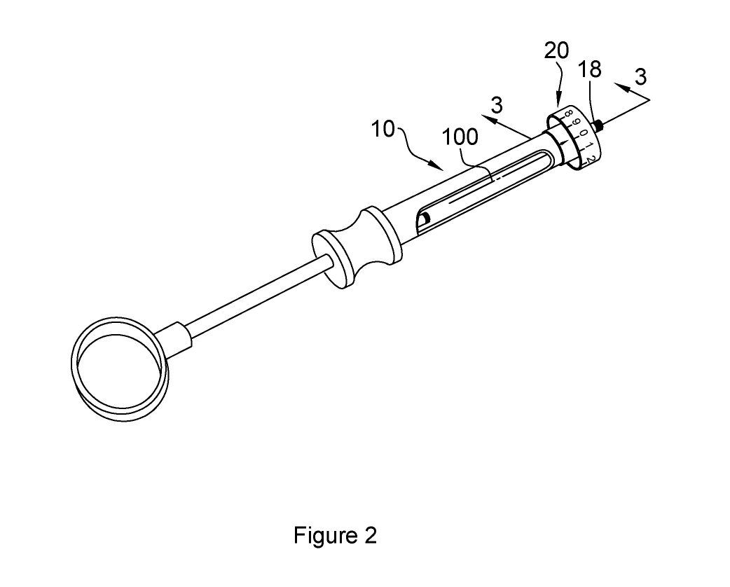

Referring to FIG. 2, an apparatus for rotating a dental needle according to a first embodiment of the invention is shown generally at 20. The apparatus 20 is adapted to be received on a standard dental syringe 10 and replaces the needle adapter 12 as shown in the prior art of FIG. 1. The apparatus 20 includes a standard external threading 18 adapted to receive a standard dental needle thereon, and rotates about a central axis 100, thereby rotating the needle during use, as will be described in more detail below.

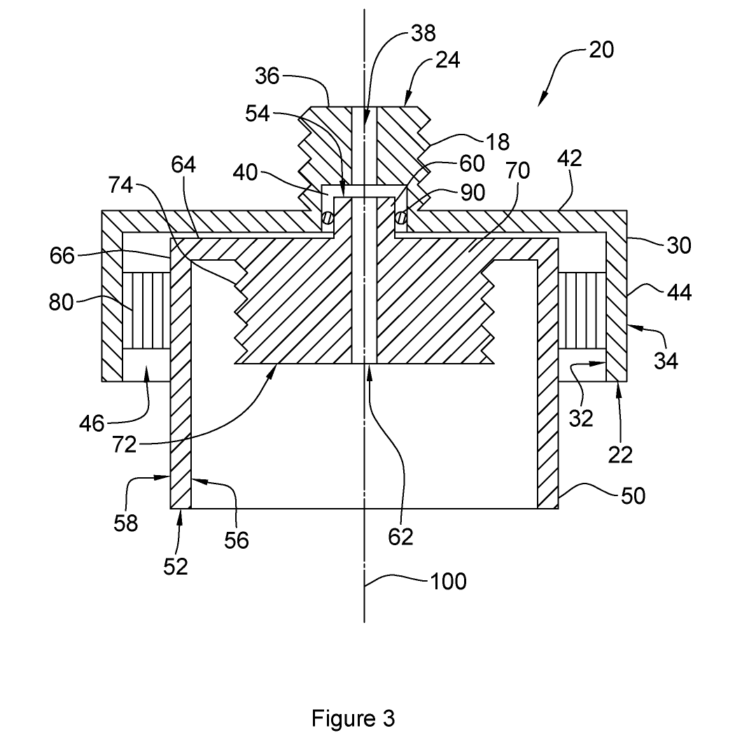

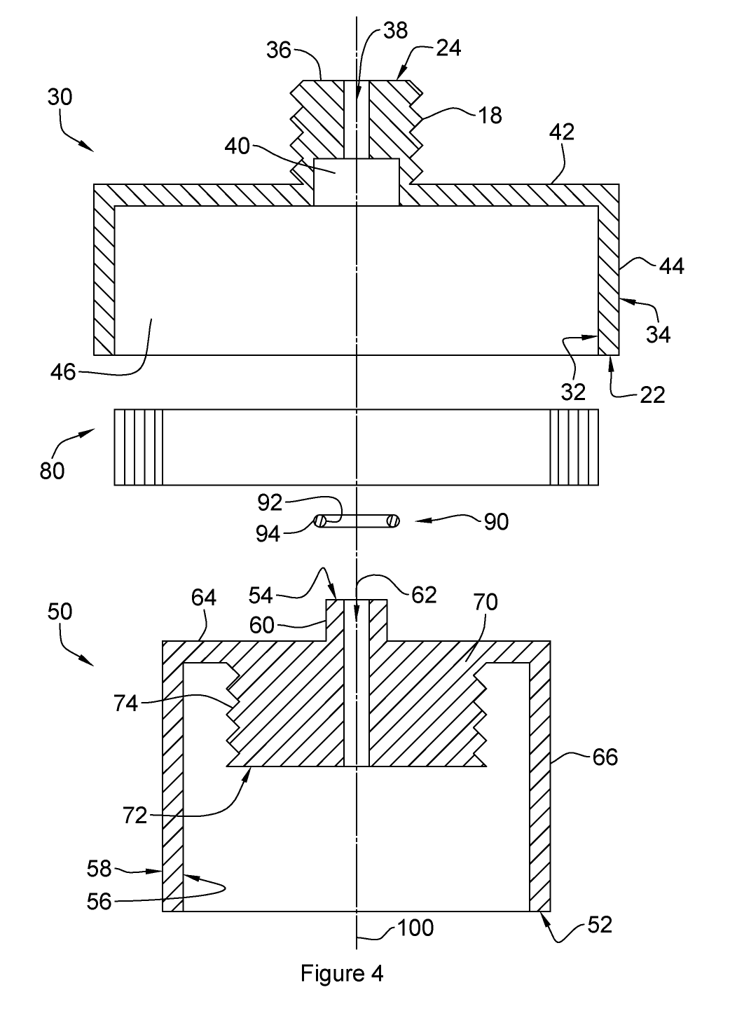

Turning now to FIGS. 3 and 4, the cylindrical apparatus 20 extends along the central axis 100. The apparatus 20 includes a cylindrical external rotating needle adapter sleeve 30 and a cylindrical internal syringe adapter 50 with a torsion spring 80 and at least one bearing 90 therebetween.

The needle adapter sleeve 30 extends between proximate and distal ends, 22 and 24, respectively, and includes inner and outer surfaces, 32 and 34, respectively. Proximate to the distal end 24, the needle adapter sleeve 30 includes a nipple portion 36 with the standard external threading 18 on the outer surface 34 adapted to receive a standard needle thereon. The nipple portion 36 includes a central bore 38 along the central axis 100 therethrough, adapted to receive a needle therethrough, as is commonly known, and a cavity 40 therein sized to receive the syringe adapter 50 with the bearing 90 therebetween. A radial wall 42 extends from the nipple portion 36 to a cylindrical sleeve portion 44. The sleeve portion 44 extends between the radial wall 42 and the proximate end 22, and includes a cavity 46 therein, sized to receive the syringe adapter 50, with the torsion spring 80 therebetween.

Referring now to FIG. 2, the needle adaptor sleeve 30 includes a plurality of indicator markings 48 on the outer surface 34. The indicator markings are spaced apart therearound and correspond to an amount of time that the needle adaptor sleeve 30 will rotate, as will be outlined in more detail below. Although sequential numbering is shown on the illustration, it will be appreciated that other indications, such as, by way of non-limiting example, number of seconds of rotation may be useful, as well.

Turning back to FIGS. 3 and 4, the syringe adapter 50 extends between proximate and distal ends 52 and 54, respectively, and includes inner and outer surfaces 56 and 58, respectively, with a central bore 62 along the central axis 100 therethrough, adapted to receive a needle therethrough, as outlined above.

Proximate to the distal end 54, the syringe adapter 50 includes a stationary nipple portion 60, contained within the cavity 40 of the nipple portion 36, with the bearing 90 between the nipple portions 60 and 36, as will be described in more detail below. A radial wall 64 extends from the nipple portion 60 to a cylindrical sleeve portion 66. The sleeve portion 66 extends between the radial wall 64 and the proximate end 52, and is sized to receive the syringe barrel 14 therein, allowing for a clearance fit therebetween.

Referring now to FIG. 2, the syringe adaptor 50 includes a reference mark 68 on the outer surface 58. When not in use, the reference mark 68 is in alignment with a start or “zero” indicator marking 48 on the needle adaptor sleeve 30. The purpose of the reference mark 68 will be set out further below. Although one reference mark 68 is indicated herein, it will be appreciated that more reference marks 68 may be useful, as well.

Turning back to FIGS. 3 and 4, a mounting portion 70 extends along the central axis 100 within the syringe adapter 50 from the radial wall 64 to a proximate end 72. The mounting portion 70 includes a standard external threading 74 thereon, adapted to engage with the internal threading 16 within the syringe barrel 14.

The spiral torsion spring 80 is secured to both the needle adapter sleeve 30 and the spring adapter 50. A plurality of gears (not shown), similar to a wind-up clock or timer or spring pull-back device, as is commonly known, may be connected thereto to control the rate at which the needle adapter sleeve spins. When wound up, the needle adaptor sleeve 30 is rotated a set number or times, then, when released, the needle adaptor sleeve 30 rotates a multiple number of times, such that the device rotates more times when unwinding compared to the number of times wound. This type of gear drive unit is commonly known, and may be such as, by way of non-limiting example, a pull-back windup type spring drive unit, although other types of gear drives may be useful, as well.

The bearing 90 includes inner and outer surfaces 92 and 94, respectively, and may be any low friction contact between the needle adapter sleeve 30 and the syringe adapter 50, such as, by way of non-limiting example, a cylindrical bearing or bushing. The bearing 90 is sized such that the outer surface 94 engages upon the inner surface 32 of the needle adapter sleeve 30 and the inner surface 92 engages upon the outer surface 58 of the syringe adapter 50. Although one bearing 90 is illustrated herein, it will be appreciated that additional bearings may be useful and may be positioned at any suitable location between the needle adapter sleeve 30 and syringe adapter 50.

To install the apparatus 20 on a syringe 10, the standard needle adapter 12 is removed and replaced with the apparatus 20. The proximate end 52 of the syringe adapter 50 is aligned over the end of the syringe 10 and rotated such that the external threading 74 engages with the internal threading 16 within the syringe barrel 14, securing the apparatus 20 thereto.

To operate the apparatus 20, a cartridge is inserted within the syringe barrel 14 and a needle is attached to the external threading 18 of the nipple portion 36 of the needle adapter sleeve 30, as is commonly known. The needle adapter sleeve 30 is rotated about the central axis 100 relative to the syringe adapter 50 to wind the torsion spring 80 in a direction generally indicated at 102. As the needle adaptor sleeve 30 is rotated, the indicator marks 48 sequentially pass by the reference mark 68. The operator may use the reference mark 68 with relation to the indicator marks 48 to visually determine the correct proportion of turns to wind the device for a set period of rotation time, similar to a rotating timer, as is commonly known. It will be appreciated that the plurality of gears between the needle adapter sleeve 30 and the spring adapter 50 may be of a design selected such that a selected wind up rotation will produce a greater unwinding rotation such as, by way of non-limiting example, as utilized in pull back wind-up toys or the like as are commonly known. When released, the needle adapter sleeve 30 rotates about the central axis 100 thereby rotating the needle. The operator then utilizes the syringe 10, as is commonly known, to inject the contents of the cartridge into the patient.

With a rotating needle, the patient experiences less tissue trauma as the needle effectively cuts the patient's tissues rather than causing the blunt force trauma of a standard stationary needle. As a result, the patient experiences less pain during and after treatment, with less bruising, and an overall better patient experience.

While specific embodiments of the invention have been described and illustrated, such embodiments should be considered illustrative of the invention only and not as limiting the invention as construed in accordance with the accompanying claims.

What is claimed is:

1. An apparatus for rotating a needle comprising:
an inner sleeve securable to a syringe barrel, said inner sleeve having an inner sleeve passage extending therethrough;
an outer sleeve rotatably supported by said inner sleeve, said outer sleeve having an outer sleeve passage extending therethrough in fluidic communication with said inner sleeve passage, said outer sleeve having a needle receptacle adapted to receive and operably couple a needle thereon;
a rotational energy-storing device, operably connected to said inner and outer sleeve so as to provide a rotational torque therebetween.

2. The apparatus of claim 1 further comprising at least one bearing rotatably supporting said outer sleeve on said inner sleeve;

3. The apparatus of claim 2 wherein said at least one bearing is selected from a group consisting of roller bearings and bushings.

4. The apparatus of claim 2 wherein said inner sleeve includes a stationary nipple extending therefrom wherein said outer sleeve includes a cavity extending thereinto wherein said stationary nipple is located within said cavity.

5. The apparatus of claim 4 wherein said at least one bearing is located around said stationary nipple within said cavity.

6. The apparatus of claim 1 wherein said inner sleeve passage and said outer sleeve passage extend along a central axis through said inner and outer sleeves.

7. The apparatus of claim 1 wherein said needle receptacle includes a nipple portion at a distal end of said outer sleeve.

8. The apparatus of claim 7 wherein said nipple portion includes external threading thereon, adapted to receive said needle thereon.

9. The apparatus of claim 8 wherein said outer sleeve passage of said outer sleeve and said inner sleeve passage of said inner sleeve are adapted to receive said needle therethrough.

10. The apparatus of claim 1 wherein said inner sleeve includes a mounting portion for securing said inner sleeve to said syringe barrel.

11. The apparatus of claim 10 wherein said mounting portion includes external threading thereon, adapted to be received within said syringe barrel.

12. The apparatus of claim 1 wherein said rotational energy storing device comprises a spiral torsion spring.

13. The apparatus of claim 12 wherein said spiral torsion spring includes first and second ends.

14. The apparatus of claim 13 wherein said first end of said spiral torsion spring is secured to an outer surface of said inner sleeve and said second end of said spiral torsion spring is secured to an inner surface of said outer sleeve.

15. The apparatus of claim 12 further comprises a gear assembly connected to said spiral torsion spring adapted to rotate said outer sleeve relative to said inner sleeve at a predefined rate.

16. A method for rotating a needle comprising:
securing an inner sleeve to a syringe barrel;
rotatably supporting an outer sleeve with said inner sleeve;
securing a needle to said outer sleeve so as to place a passage through said needle in fluidic communication with a capsule within said syringe barrel;
rotating said outer sleeve relative to said inner sleeve so as to store rotational energy within a rotational energy-storing device extending between said inner and outer sleeves;
releasing said outer sleeve such that said needle rotates relative to said syringe barrel.
